(19) United States
(12) Patent Application Publication
(10) Pub. No.: US 2023/0000929 A1
(43) Pub. Date: Jan. 5, 2023

(54) BDELLOVIBRIO TREATMENT FOR AMYOTROPHIC LATERAL SCLEROSIS

(71) Applicant: Board of Regents, the University of Texas System, Austin, TX (US)

(72) Inventors: Andrew M. PICKERING, San Antonio, TX (US); Sang Woo KIM, San Antonio, TX (US)

(21) Appl. No.: 17/780,436

(22) PCT Filed: Nov. 19, 2020
(86) PCT No.: PCT/US2020/061158

§ 371 Date: May 26, 2022

Related U.S. Application Data
(60) Provisional application No. 62/940,421, filed on Nov. 26, 2019.

(51) Int. Cl. A61K 35/74; A61P 25/28; A23L 33/10; A61K 35/741; A61K 9/48; A61K 9/00

(52) U.S. Cl. CPC A61K 35/74; A61P 25/28; A23L 33/10; A61K 35/741; A61K 9/48; A61K 9/0053; A61K 9/0031

(57) ABSTRACT

Compositions and methods for treating or preventing the progression of neurodegenerative diseases are provided herein. Exemplary compositions include bacterial compositions having an effective amount of viable, non-pathogenic microbes, viable, non-pathogenic bacteria, wherein at least one of the bacteria is a predatory bacteria such as Bdellovibrio bacteriovorus. The disclosed bacterial compositions can be used to treat or prevent the progression of neurodegenerative diseases such as ALS, Alzheimer's disease, Huntington's disease, and Parkinson's disease.

### TECHNICAL FIELD OF THE INVENTION

This invention is generally related to compositions and methods of treating neurodegenerative diseases.

### BACKGROUND OF THE INVENTION

Neurodegenerative diseases are incurable, debilitating conditions that are characterized by the progressive degeneration and death of nerve cells. Neurons are the building blocks of the nervous system. The loss or dysfunction of neurons in patients with neurodegenerative disease can affect body movement and brain function because neurons do not usually reproduce or replace themselves when they become damaged or die. Common neurodegenerative diseases include but are not limited to Alzheimer's disease, amyotrophic lateral sclerosis (ALS), Huntington's disease, Parkinson's disease, Prion disease, motor neuron disease, spinocerebellar ataxia, and spinal muscular atrophy. The symptoms of advanced neurodegenerative diseases can be devastating, with patients losing their memory, control over their movements, and their personality.

The pathogenesis of these conditions remains unclear, making it difficult to find targets for new treatments. There are currently no cures for neurodegenerative diseases and treatments predominantly focus on treating the symptoms associated with the diseases. These treatments typically confer negative side effects to the patient that further deteriorate their quality of life. Recent clinical trials have shown some early promise of immunotherapy to slow disease progression; however, this therapy is both in early development and highly expensive.

Recent studies suggest that the pathophysiology of some neurodegenerative diseases such as ALS and Parkinson's disease may be linked to the gastrointestinal tract. The gut microbiota is sometimes referred to as the second brain. It affects brain activity through the gut-microbiota-brain axis (Martin, et al., Cell Mol Gastroenterol Hepatol, 6:133-148 (2018)). Gut microbes communicate to the central nervous system through nervous, endocrine, and immune signaling mechanisms. Several microbially derived molecules play an important role in nervous system modulation, namely short-chain fatty acids, secondary bile acids, and tryptophan metabolites. These molecules propagate signals primarily through interaction with intestinal epithelial cells such as enteroendocrine cells and enterochromaffin cells, and the mucosal immune system, but some cross the intestinal barrier, enter systemic circulation, and may cross the blood-brain barrier. Small intestinal dysbiosis and increased intestinal permeability are common in patients with some neurodegenerative diseases (Fang, X, Int J Neurosci, 126:771-776 (2016)). Following intestinal epithelial barrier disruption, gut microbiota-derived products, such as LPS and other neurotoxins, can cross the intestinal barrier into the blood stream. LPS and other neurotoxins cause systemic inflammatory responses which are thought to play pivotal roles in the pathogenesis of neurodegenerative diseases. Therefore, repairing or protecting the intestinal barrier, or neutralizing pathogens in circulation could prevent the pathogenesis of neurodegenerative diseases.

Therefore, it is an object of the invention to provide compositions and methods of treating and preventing the progression of neurodegenerative diseases.

### SUMMARY OF THE INVENTION

Bacterial compositions and methods of their use for treating and preventing the progression of neurodegenerative diseases are provided herein. Exemplary methods include administering to the subject an effective amount of a bacterial composition including an effective amount of at least one predatory bacterial species. In one embodiment, the predatory bacteria is Bdellovibrio bacteriovorus. The bacterial composition can be administered to the subject once daily, twice daily, or three times daily. The neurodegenerative disease that is treated can be amyotrophic lateral sclerosis (ALS), Alzheimer's disease, Huntington's disease, and Parkinson's disease.

Another embodiment provides a method of prophylactically reducing or inhibiting the progression of neurodegenerative disease in a subject in need thereof by administering to the subject a bacterial composition including at least one predatory bacterial species in an amount to reduce or inhibit the progression of a neurodegenerative disease. In some embodiments, the subject has a family history of neurodegenerative disease.

Also provided is a method for treating inflammation of the gastrointestinal system in a subject in need thereof by administering to the gastrointestinal system of the subject an effective amount of Bdellovibrio bacteriovorus. In one embodiment, the subject has or is suspected of having a neurodegenerative disease, for example ALS.

Exemplary bacterial compositions include an effective amount of at least one predatory bacterial species. In one embodiment, the predatory bacteria is Bdellovibrio bacteriovorus. The bacterial composition can include a concentration of 10⁶ to 10¹² CFU of predatory bacteria. In some embodiments, the Bdellovibrio bacteriovorus are genetically engineered to target specific pathogenic microbes.

In some embodiments, the bacterial composition also includes prebiotic compositions. Exemplary prebiotics include inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, pectin, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol. In one embodiment, the prebiotic is dietary fiber.

In another embodiment, the bacterial composition also includes one or more viable commensal microbes. Exemplary commensal microbes include Bifidobacterium bifidum, Bifidobacterium breve, Bifidobacterium infantis, Bifidobacterium lactis, Bifidobacterium longum, Lactobacillus acidiophilis, Lactobacillus brevis, Lactobacillus bulgaricus, Lactobacillus casei, Lactobacillus fermentum, Lactobacilluis gasseri, Lactobacillus lantarum, Lactobacillus paracasei, Lactobacillus reuteri, Lactobacillus rhamnosus, Lactobacillus salivarius, Lactococcus lactis, Streptococcus thermophilia, Bacillus coagulans, Bacillus laterosporus, Pediococcus acidilactici, and Saccharomyces boulardii.

The bacterial compositions can be formulated for enteral administration, including but not limited to enteral administration. The composition can be an extended release composition such as a time controlled capsule, a pH controlled capsule or an enzyme controlled capsule. The bacterial composition can also be formulated as a supplemental drink or in a food stuff, for example yogurt. In other embodiments, the bacterial composition is formulated for rectal administration.

### BRIEF DESCRIPTION OF THE DRAWINGS

FIG. 1A is a dot plot showing microbial diversity in patients with spinal-onset ALS, bulbar-onset ALS, or healthy (control) patients. The data is shown as alpha diversity measure. FIG. 1B is a dot plot showing operational taxonomic unit (OTU) richness in the microbiome of patients with spinal-onset ALS, bulbar-onset ALS, or healthy (control) patients. FIG. 1C is a heat map showing microbiome clustering in patients with spinal-onset ALS, bulbar-onset ALS, or healthy (control) patients. FIG. 1D is a dot plot showing the prevalence of Bdellovibrio in the microbiome of patients with spinal-onset ALS, bulbar-onset ALS, or healthy (control) patients. The Bdellovibrio prevalence is expressed as a percentage of the while microbiome.

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “pharmaceutically acceptable” means a non-toxic material that does not interfere with the effectiveness of the biological activity of the active ingredients.

The term “pharmaceutically-acceptable carrier” means one or more compatible solid or liquid fillers, dilutants or encapsulating substances which are suitable for administration to a human or other vertebrate animal.

The term “effective amount” or “therapeutically effective amount” means a dosage sufficient to provide treatment a disorder, disease, or condition being treated, or to otherwise provide a desired pharmacologic and/or physiologic effect. The precise dosage will vary according to a variety of factors such as subject-dependent variables (e.g., age, immune system health, etc.), the disease, and the treatment being effected.

The terms “individual,” “individual,” “subject,” and “patient” are used interchangeably herein, and refer to a mammal, including, but not limited to, humans, rodents, such as mice and rats, and other laboratory animals.

As used herein, “probiotics” are live bacteria or yeast that when consumed confer a health benefit to the host. Probiotics are said to restore the balance of bacteria in the gut when it has become disrupted through long-term antibiotic use or gastrointestinal disease. Examples of microbial species typically used in probiotics include but are not limited to Bifidobacterium bifidum, Bifidobacterium breve, Bifidobacterium infantis, Bifidobacterium lactis, Bifidobacterium longum, Lactobacillus acidiophilis, Lactobacillus brevis, Lactobacillus bulgaricus, Lactobacillus casei, Lactobacillus fermentum, Lactobacilluis gasseri, Lactobacillus lantarum, Lactobacillus paracasei, Lactobacillus reuteri, Lactobacillus rhamnosus, Lactobacillus salivarius, Lactococcus lactis, Streptococcus thermophilia, Bacillus coagulans, Bacillus laterosporus, Pediococcus acidilactici, and Saccharomyces boulardii.

As used herein, a “prebiotic” is a selectively fermented ingredient that allows specific changes, both in the composition and/or activity of the gastrointestinal microflora that confers benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol.

As used herein, a “synbiotic” is a product that contains both a probiotic and a prebiotic.

As used herein, the term “microbiota” refers to the community of microorganisms such as bacteria, archaea, fungi, and viruses that inhabit an ecosystem or organism. The synonymous term “microbiome” refers to the microorganisms or the collective genomes of the microorganisms that reside in an environmental niche.

As used herein, the terms “gut microbiome”, “intestinal microbiome” are interchangeable and are intended to represent the normal, naturally occurring bacterial population present in the gastric and intestinal systems of healthy humans and animals. It is meant to reflect both the variety of bacterial species and the concentration of bacterial species found in a healthy human or animal.

As used herein, the term “CFU” means colony forming unit and refers to the amount of bacteria in a probiotic that are viable and capable of dividing and forming colonies.

The terms “treat,” “treating,” or “treatment” refer to alleviating, reducing, or inhibiting one or more symptoms or physiological aspects of a disease, disorder, syndrome, or condition. “Treatment” as used herein covers any treatment of a disease in a subject, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

As used herein, the term “motor neuron” refers to a neuron whose cell body is located in the motor cortex, brainstem, or spinal cord, and whose axon projects to the spinal cord or outside of the spinal cord to directly or indirectly control effector organs, mainly muscles and glands.

As used herein, the term “predatory microbes” and “predatory bacteria” refer to microorganisms that prey upon other microorganisms that are pathogenic to humans.

### II. Methods of Treating Amyotrophic Lateral Sclerosis and Other Neurodegenerative Diseases

Methods of treating, inhibiting or reducing the progression of neurodegenerative diseases are provided herein. Methods typically include administering an effective amount of a bacterial composition including at least one predatory bacterium to a subject in need thereof. In one embodiment, the predatory bacterium is Bdellovibrio bacteriovorus. In some embodiments, the disclosed methods reduce symptoms of neurodegenerative diseases and slow the progression of the diseases.

It has been reported that patients with ALS disease experience elevated gut leakage resulting in an increase in prevalence of microbes in the blood stream which can contribute to immune response and inflammatory signaling, potentially accelerating disease progression (Fang 2016). Patients with other neurodegenerative diseases, such as Parkinson's disease, have also been reported to have intestinal epithelial dysfunction. Without being bound to any one theory, it is thought that treatment with predatory bacteria, such as Bdellovibrio bacteriovorus, will reduce the presence of harmful microbiota in the blood system of patients with ALS, reduce inflammation and slow disease progression. In one embodiment, the predatory bacteria kill bacteria that have leaked from the intestines into circulation.

One embodiment provides a method of treating neurodegenerative disease in a subject in need thereof by administering to the subject an effective amount of a predatory bacteria to treat the disease. In one embodiment, at least one predatory bacteria is Bdellovibrio bacteriovorus. In one embodiment, the predatory bacteria reduces the amount of pathogenic microbes in the circulation of a subject in need thereof. Reducing the number of pathogenic microbes can reduce systemic inflammation and the progression of neurodegenerative diseases.

Another embodiment provides a method for treating inflammation of the gastrointestinal system in a subject in need thereof by administering to the gastrointestinal system of the subject an effective amount of Bdellovibrio bacteriovorus. In one embodiment, the subject has or is suspected of having a neurodegenerative disease.

In one embodiment, the disclosed bacterial compositions are administered to a subject on a daily basis. The bacterial compositions can be administered to a subject once daily, twice daily, or three times daily. In another embodiment, the bacterial compositions are administered to a subject every other day. In other embodiments, the bacterial compositions are administered to a subject once weekly, twice weekly, or three times weekly.

### A. Subjects to Be Treated

### 1. Amyotrophic Lateral Sclerosis (ALS)

In one embodiment, the disclosed bacterial compositions can treat, inhibit or reduce amyotrophic lateral sclerosis (ALS). ALS, also called Lou Gehrig's disease, is a progressive neurodegenerative disease that affects motor neurons in the brain and spinal cord. Symptoms of ALS include but are not limited to difficulty speaking, swallowing, walking, moving, and breathing. ALS usually affects men and women between the ages of 40 to 70. There are two different types of ALS, sporadic and familial. Sporadic, which is the most common form of the disease in the U.S., accounts for 90 to 95 percent of all cases.

Approximately two thirds of patients with typical ALS have a spinal form of the disease (also referred to as classical ‘Charcot ALS’ or spinal-onset ALS). Initial symptoms of this form include muscle weakness or wasting in the arms and legs, and involuntary muscle contractions resulting in twitches. Patients with spinal-onset ALS tend to progress to paralysis or death within three to five years. In one embodiment, the disclosed bacterial compositions can be administered to patients with spinal-onset ALS to prevent or slow disease progression.

In patients with bulbar onset ALS, the muscles involved in speaking, swallowing, and breathing are generally the first to be affected. Initial symptoms include slurry speech and difficulty swallowing. With bulbar ALS, patients may be paralyzed within one to two years. In one embodiment, the disclosed bacterial compositions can be administered to patients with bulbar-onset ALS to prevent or slow disease progression.

One embodiment provides a method of treating ALS in a subject in need thereof by administering a bacterial composition including an effective amount of at least one predatory bacteria to a subject in need thereof. In one embodiment, at least one predatory bacteria is Bdellovibrio bacteriovorus. In some embodiments, the composition includes prebiotic material, commensal gut microbiota, or a combination thereof.

Other motor neuron diseases that can be treated using the disclosed bacterial compositions include but are not limited to progressive bulbar palsy, pseudobulbar palsy, primary lateral sclerosis, spinal muscular atrophy, and post-polio syndrome.

### 2. Parkinson's Disease

Parkinson's disease is a neurodegenerative disorder that predominantly affect dopamine-producing neurons in a specific area of the brain called substantia nigra. Parkinsons's disease is a progressive disease that worsens over time as more neurons become impaired or die. The cause of neuronal death in Parkinson's is not known. Symptoms of Parkinson's disease include but are not limited to tremors in hands, arms, legs, jaw, or head, stiffness of the limbs and trunk, slowness of movement, and impaired balance and coordination.

One embodiment provides a method of treating Parkinson's disease by administering a bacterial composition including at least one predatory bacteria to a subject in need thereof in an amount effective to reduce symptoms of Parkinson's disease and slow the progression of the disease. In one embodiment, at least one predatory bacteria is Bdellovibrio bacteriovorus. In some embodiments, the composition includes prebiotic material, commensal gut microbiota, or a combination thereof.

In one embodiment, the disclosed bacterial compositions can be administered to a subject prophylactically if the subject has a family history of Parkinson's disease or other neurodegenerative diseases. The bacterial compositions can inhibit or reduce the onset of Parkinson's disease and symptoms thereof.

### 3. Huntington's Disease

Huntington's disease is a progressive neurodegenerative disease. The disease is characterized by the progressive breakdown of nerve cells in the brain. Symptoms of

Huntington's disease include but are not limited to involuntary movement problems and impairments in voluntary movement such as involuntary jerking, muscle rigidity, slow or abnormal eye movements, impaired gait, posture, and balance, difficulty with the physical production of speech or swallowing; cognitive impairments such as difficulty organizing, prioritizing, or focusing on tasks, lack of flexibility or the tendency to get stuck on a thought, behavior, or action, lack of impulse control, lack of awareness of one's own behaviors and abilities, slowness in processing thoughts or finding words, and difficulty in learning new information; and psychiatric disorders such as depression. In one embodiment, the disclosed bacterial compositions can lessen or slow down the progression of symptoms of Huntington's disease.

One embodiment provides a method of treating Huntington's disease in a subject in need thereof by administering bacterial including an effective amount of a predatory bacteria to the subject to reduce systemic inflammation and protect neurons. In one embodiment, at least one predatory bacteria is Bdellovibrio bacteriovorus. In one embodiment, bacterial compositions slow down or inhibit the progression of disease symptoms in subjects with Huntington's disease.

In some embodiments, the composition includes prebiotic material, commensal gut microbiota, or a combination thereof.

Huntington's disease is largely genetic, every child of a parent with Huntington's disease has a 50/50 chance of inheriting the disease. In one embodiment, subjects with a familial history of Huntington's disease can be prophylactically administered one of the disclosed bacterial compositions to inhibit or reduce the onset of Huntington's disease and symptoms thereof.

### 4. Alzheimer's Disease

Alzheimer's disease is a progressive disorder that causes brain cells to degenerate and eventually die. Alzheimer's disease is the most common cause of dementia—a continuous decline in thinking, behavioral and social skills that disrupts a person's ability to function independently. Symptoms of Alzheimer's disease include but are not limited to memory loss, impairment in thinking and reasoning abilities, difficulty in making judgments and decisions, and changes in personality and behavior. While the exact cause of Alzheimer's disease is not fully understood, it is believed that the core problem is dysfunctionality in brain proteins which disrupt neuronal function and unleash a series of toxic events. The damage most often starts in the region of the brain that controls memory, but the process begins years before the first symptoms. The loss of neurons spreads in a somewhat predictable pattern to other regions of the brains. By the late stage of the disease, the brain has shrunk significantly. Beta-amyloid plaques and tau protein tangles are most often attributed with the bulk of the damage and dysfunctionality of neurons in Alzheimer's disease.

One embodiment provides a method of treating Alzheimer's disease in a subject by administering a bacterial composition including at least one predatory bacteria to the subject in an amount effective to reduce systemic inflammation and protect motor neurons. In one embodiment, at least one predatory bacteria is Bdellovibrio bacteriovorus. In another embodiment, subjects are administered an effective amount of the disclosed bacterial compositions to reduce or eliminate symptoms of Alzheimer's disease or to slow down disease progression. In some embodiments, the composition includes prebiotic material, commensal gut microbiota, or a combination thereof.

In one embodiment, subjects with a family history of Alzheimer's disease can be prophylactically administered one or more of the disclosed bacterial compositions to inhibit or reduce the onset of Alzheimer's disease.

### III. Bacterial Compositions

Compositions for treating, reducing, or inhibiting the progression of neurodegenerative diseases in a subject are provided. One embodiment provides a bacterial composition containing an effective amount of at least one predatory bacteria. The composition can also include other commensal microbes and/or prebiotics. The disclosed probiotics can be administered to a subject in an amount effective to treat or prevent the progression of neurodegenerative diseases.

### A. Predatory Bacteria

Bacterial compositions containing one more predatory bacteria are disclosed that can treat, inhibit or reduce the progression of neurodegenerative diseases. Predatory bacteria prey upon other microbes that are pathogenic to humans but do not predate human cells. Exemplary predatory bacteria that can be included in the disclosed bacterial compositions include but are not limited to Bacteriovorax starrii, Bacteriovax stolpii, Bdellovibrio bacteriovorus, Myxococcus, Lysobacter, Aristabacter necator, Bdellovibrio exovorus, Bdellovibrio starrii, Bdellovibrio stolpii and M. xanthus. In one embodiment, the predatory bacteria is Bdellovibrio bacteriovorus. The concentration of Bdellovibrio bacteriovorus to be used in the bacterial composition is about 10⁶ to 10¹² CFU. In one embodiment, a concentration of 10⁶, 10⁷, 10⁸, 10⁹, 10¹⁰, 10¹¹, or 10¹² CFU of Bdellovibrio bacteriovorus.

In one embodiment, the Bdellovibrio bacteriovorus can be genetically altered to target specific microbes. Predatory bacteria naturally and obligately prey on other Gram negative bacteria. In one embodiment, the Bdellovibrio bacteriovorus can be genetically altered to prey upon Gram positive bacteria, for example the Bdellovibrio bacteriovorus can be altered to recognize and target the outer peptidoglycan layer of Gram-positive bacteria. In another embodiment, the Bdellovibrio bacteriovorus are genetically altered to sense endotoxins or enzymes secreted from pathogenic bacteria. The Bdellovibrio bacteriovorus can be engineered to target microbes that are involved in the pathogenesis of neurodegenerative diseases. Exemplary pathogenic microbes include but are not limited to Bacteroides, Campylobacter, Clostridium, Enterobacteria, Escherichia coli, Shigella, and Salmonella. In another embodiment, the Bdellovibrio bacteriovorus are engineered to target and kill antibiotic resistant bacteria.

### B. Commensal Bacteria

In some embodiments, the disclosed bacterial compositions contain commensal bacteria. Representative commensal bacteria that can be included in the disclosed bacterial compositions include but are not limited to Bifidobacterium bifidum, Bifidobacterium breve, Bifidobacterium infantis, Bifidobacterium lactis, Bifidobacterium longum, Lactobacillus acidiophilis, Lactobacillus brevis, Lactobacillus bulgaricus, Lactobacillus casei, Lactobacillus fermentum, Lactobacilluis gasseri, Lactobacillus lantarum, Lactobacillus paracasei, Lactobacillus reuteri, Lactobacillus rhamnosus, Lactobacillus salivarius, Lactococcus lactis, Streptococcus thermophilia, Bacillus coagulans, Bacillus laterosporus, Pediococcus acidilactici, and Saccharomyces boulardii. The concentration of other probiotics to include in the bacterial composition is 10⁴ to 10¹⁰ CFU. In one embodiment, the bacterial composition contains 100 million CFU of each bacterial species per gram.

### C. Prebiotic Compositions

In some embodiments, the bacterial composition includes a prebiotic. Prebiotics are selectively fermented ingredients that stimulate the growth and/or activity of one or a limited number of bacteria in the gastrointestinal flora that confers benefits upon host well-being and health. Examples of prebiotics include but are not limited to inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, pectin, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol. Bacterial compositions can contain a daily dose of prebiotics in the range 5 g-20 g.

In one embodiment, the bacterial composition contains dietary fiber. Dietary fiber is the indigestible portion of food produced by plants. It has a wide-range of health benefits including lower risk of heart disease and maintenance of gut health. Dietary fiber can be included in the bacterial composition disclosed herein at a daily range of 2.5 g-5 g.

### D. Pharmaceutical Compositions

The disclosed bacterial compositions can be formulated as pharmaceutical compositions. The pharmaceutical compositions containing the bacterial compositions can be formulated for enteral administration including oral or rectal routes of administration. The disclosed bacterial compositions are typically administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed bacterial compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed bacterial compositions, the bacterial concentrations also depend on the type of bacterium. For the disclosed bacterial compositions, generally dosage levels of 10⁸-10¹² CFU daily are administered to mammals.

### 1. Formulations for Enteral Administration

Oral solid dosage forms are described generally in Remington Essentials of

Pharmaceutics, 1st Ed. 2013 (Pharmaceutical Press, London) at Chapter 30. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the disclosed. See, e.g., Remington Essentials of Pharmaceutics, 1st Ed. 2013 (Pharmaceutical Press, London) Chapter 37 which are herein incorporated by reference. The compositions may be prepared in liquid form, or may be in dried powder (e.g., lyophilized) form. Liposomal or proteinoid encapsulation may be used to formulate the compositions. Liposomal encapsulation may be used and the liposomes may be derivatized with various polymers (e.g., U.S. Pat. No. 7,108,863) In general, the formulation will include the bacterial composition and inert ingredients which protect peptide in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations are also provided. The bacterial compositions can be incorporated into an inert matrix which permits release by either diffusion or leaching mechanisms, e.g., gums. Slowly degenerating matrices may also be incorporated into the formulation. Another form of a controlled release is based on the The osmotic-controlled release oral delivery system (OROS™) (Alza Corp.), i.e., the drug is enclosed in a semipermeable membrane which allows water to enter and push drug out through a single small opening due to osmotic effects.

For a bacterial to successfully exert its benefit on the host's gut microbiota it should be able to remain viable during storage and also be capable of surviving, and potentially colonizing, the host's intestinal environment. Therefore, the bacterial composition should contain a concentration of live bacteria that is effective in causing benefits in the subject. Additionally, the capsule, pill, tablet, or syrup for oral administration should be stored in a manner so as to preserve its efficacy. Methods of storage include but are not limited to refrigeration, freezing, or storing at room temperature. If stored at room temperature, the bacterial should be stored in an air tight container.

### 2. Targeted Delivery

For enteral formulations, the location of release may be the small intestine (the duodenum, the jejunum, or the ileum), or the large intestine. The contents will be delivered via a method that ensures proper transit and survival to the targeted portion of the GI tract, depending on the targeted disease or disorder. For example, any products designed to treat or prevent Clostridium difficile infection will be formulated in a colon-targeted capsule, such as DRcaps™ from Capsugel®. Probiotics exert their main effect in the intestinal tract so in some embodiments, the release will avoid the deleterious effects of the stomach environment, either by protection of the agent (or derivative) or by release of the agent (or derivative) beyond the stomach environment. To ensure full gastric resistance, a coating impermeable to at least pH 5.0 is essential. Examples of common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D™, Aquateric™, cellulose acetate phthalate (CAP), Eudragit L™, Eudragit S™, and Shellac™. These coatings may be used as mixed films.

### a. Delayed Release Formulation

Delayed release formulations can be created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### i. Time Controlled Capsules

In one embodiment, the bacterial compositions are formulated into time controlled capsules. The capsules may be manufactured using erodible capsule coating or specialized internal fillings so that the contents are released following transit of the capsule from the stomach to the upper or lower intestines. Time controlled release systems utilize measurements of gastric emptying and intestinal motility to ensure that the active ingredient stays protected until the capsule has cleared the stomach.

### ii. pH Controlled Capsules

In one embodiment, the bacterial compositions are formulated into pH controlled capsules. The pH in the stomach ranges from 1 to 2 during fasting but increases after eating. In the small intestine, the pH is around 6.5 in the proximal regions and increases to about 7.5 in the distal portions. From there, the pH declines significantly as intestinal contents reach the cecum and then the colon, pH 6.4 and 5.7 respectively. These pH differences between portions of the GI tract can be utilized for the formulation of encapsulations based on polymers that are insoluble in low pH environments (e.g., the stomach) and soluble in higher pH environments (e.g. the lower digestive tract) (Philip & Philip, Oman Medical Journal, 25:79-87 (2003)).

### iii. Enzyme Controlled Capsules

In some embodiments, the bacterial compositions are formulated into enzyme controlled capsules. Microbes that are specific to different portions of the GI tract are constantly fermenting the contents of their environment to generate the energy needed for their survival. Byproducts of the fermentation process include enzymes that can be specific to the microbe and the substance they are fermenting. As a result, capsules that will degrade only in the presence of site-specific enzymes can be designed (Philip & Philip, Oman Medical Journal, 25:79-87 (2003)).

### IV. Co-Therapies

The disclosed bacterial compositions can be administered with a second therapeutic that is selected based on the subject's disease state. The second therapeutic can be a treatment for Alzheimer's disease. Current treatments for Alzheimer's disease include but are not limited to cholinesterase inhibitors such as donepezil, rivastigmine, and galantamine; memantine; antidepressants such as citalopram, fluoxetine, paroxetine, sertraline, and trazadone; anxiolytics such as lorazepam and oxazepam; and antipsychotics such as aripiprazole, clozapine, haloperidol, olanzapine, quetiapine, risperidone, and ziprasidone.

In another embodiment, the additional therapeutic agent can be a treatment for ALS. There are currently two FDA approved treatments for ALS, riluzole and edavarone. Both drugs have been shown to slow down the progression of ALS. In addition to riluzole and edavarone, subjects with ALS can also be treated with drugs that target a specific symptom of the disease. Exemplary drugs include but are not limited to drugs to reduce spasticity such as antispastics like baclofen, dantrolene, and diazepam; drugs to help control nerve pain such as amitriptyline, carbamazepine, duloxetine, gabapentin, lamotrigine, milnacipran, nortriptyline, pregabalin and venlafaxine; and drugs to help patients swallow such as trihexyphenidyl or amitriptyline.

In one embodiment, the additional therapeutic agent can be a treatment for Parkinson's disease. Current treatments for Parkinson's disease include but are not limited to carbidopa-levodopa; dopamine agonists such as pramipexole, ropinirole, and rotigotine; MAO B inhibitors such as selegiline, rasagiline, and safinamide; catechol O-methyltransferase inhibitors such as entacapone and tolcapone; anticholinergics such as bentztropine and trihexyphenidyl; and amantadine.

The second therapeutic agent can be a treatment for Huntington's disease. Current treatments for Huntington's disease include but are not limited to tetrabenazine; antipsychotics such as haloperidol, chlorpromazine, risperidone, and quetiapine; amantadine; levetiracetam; clonazepam; antidepressants such as citalopram, escitalopram, fluoxetine, and sertraline; and anticonvulsants such as valproate, carbamazepine, and lamotrigine.

### EXAMPLES

### Example 1. Bdellovibrio Decreased in Microbiome of ALS Patients

### Materials and Methods

Stool samples and saliva samples were collected from 15 patients with Spinal onset ALS disease, 6 patients with Bulbar-onset ALS disease and 7 controls. Genomic DNA was isolated from these samples and sequenced by the company Seqmatic (Fremont, Calif.). Following sequencing an open reference operational taxonomic unit (OTU) picking strategy was employed. In this approach all sequences were assigned to a strain OTU and compared with a reference database. The resultant informed both on the microbiome makeup of the sample along with the prevalence of individual strains.

### Results

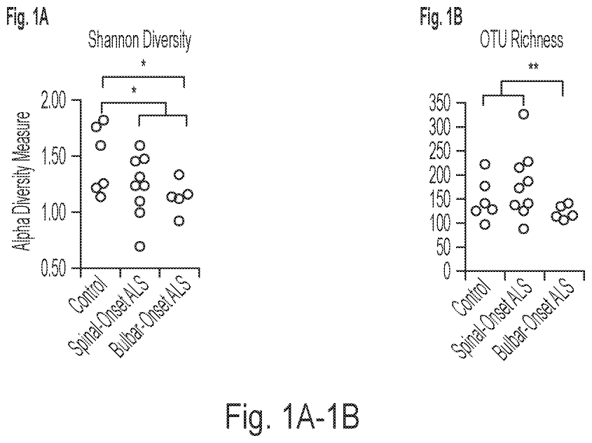

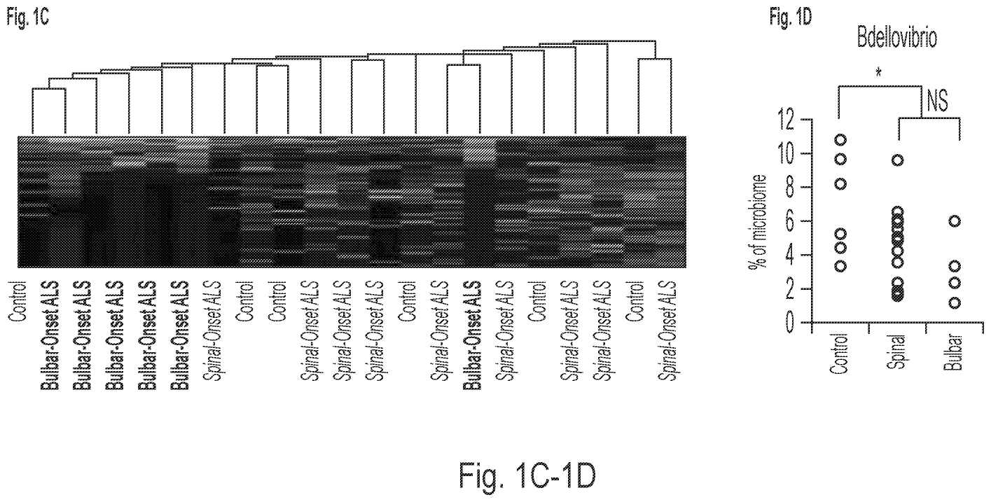

The results showed a significant decline in Shannon diversity, with a trend for reduced OTU richness in patients with ALS disease. This trend appeared strongest in patients with the more severe Bulbar-Onset variant (FIGS. 1A-1B). It was further demonstrated that microbiome cluster significantly in patients with bulbar-onset ALS disease relative to control and spinal-onset patients (FIG. 1C). One of the most striking differences in microbiome alterations was a significant decline in Bdellovibrio in patients with ALS disease particularly those with bulbar-onset ALS disease (FIG. 1D).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for treating neurodegenerative diseases in a subject in need thereof comprising, administering to the subject an effective amount of a bacterial composition comprising an effective amount of at least one predatory bacterial species.

2. The method of claim 1, wherein the predatory bacterial species is Bdellovibrio bacteriovorus.

3. The method of claim 1, wherein the effective amount of bacteria is 10⁶ to 10¹² CFU.

4. (canceled)

5. The method of claim 1, wherein the bacterial composition further comprises prebiotic compositions is selected from the group containing inulin, arabinoxylan, xylose, soluble fiber dextran, soluble corn fiber, polydextrose, lactose, N-acetyl-lactosamine, glucose, galactose, fructose, rhamnose, mannose, uronic acids, 3′-fucosyllactose, 3′-sialylactose, 6′-sialyllactose, lacto-N-neotetraose, 2′-2′-fucosyllactose, trans-galactooligosaccharides, glucooligosaccharides, isomaltooligosaccharides, lactosucrose, polydextrose, pectin, soybean oligosaccharides, and arabinose, cellobiose, fructose, fucose, galactose, glucose, lactose, lactulose, maltose, mannose, ribose, sucrose, trehalose, xylobiose, xylooligosaccharide, D-xylose, and xylitol.

6. The method of claim 5, wherein the prebiotic is dietary fiber.

7. (canceled)

8. The method of claim 1, wherein the bacterial composition further comprises one or more viable commensal microbes are selected from the group consisting of Bifidobacterium bifidum, Bifidobacterium breve, Bifidobacterium infantis, Bifidobacterium lactis, Bifidobacterium longum, Lactobacillus acidiophilis, Lactobacillus brevis, Lactobacillus bulgaricus, Lactobacillus casei, Lactobacillus fermentum, Lactobacilluis gasseri, Lactobacillus lantarum, Lactobacillus paracasei, Lactobacillus reuteri, Lactobacillus rhamnosus, Lactobacillus salivarius, Lactococcus lactis, Streptococcus thermophilia, Bacillus coagulans, Bacillus laterosporus, Pediococcus acidilactici, and Saccharomyces boulardii.

9. The method of claim 1, wherein the bacterial composition is formulated for oral administration.

10. The method of claim 9, wherein the bacterial composition is formulated as a time controlled capsule, a pH controlled capsule, or an enzyme controlled capsule.

11. (canceled)

12. (canceled)

13. The method of claim 1, wherein the bacterial composition is formulated for rectal administration.

14. The method of claim 1, further comprising administering a second therapeutic agent.

15. The method of claim 1, wherein the neurodegenerative disease is selected from the group consisting of amyotrophic lateral sclerosis (ALS), Alzheimer's disease, Huntington's disease, and Parkinson's disease.

16. The method of claim 1, wherein the bacterial composition is administered once daily, twice daily, or three times daily.

17. The method of claim 1, for treating inflammation of the gastrointestinal system in a subject in need thereof comprising administering to the gastrointestinal system of the subject an effective amount of Bdellovibrio bacteriovorus.

18. The method of claim 17, wherein an effective amount of Bdellovibrio bacteriovorus is administered to the gastrointestinal system of the subject that has or is suspected of having a neurodegenerative disease.

19. The method of claim 17, wherein the neurodegenerative disease is ALS.

20. A method of prophylactically treating neurodegenerative disease in a subject in need thereof comprising administering to the subject a bacterial composition comprising at least one predatory bacterial species in an amount effective to inhibit or reduce the onset of a neurodegenerative disease.

21. The method of claim 19, wherein the subject in need thereof has a family history of neurodegenerative disease.

22. A bacterial composition for the treatment of neurodegenerative diseases comprising an effective amount of viable, non-pathogenic bacteria, wherein at least one of the bacteria is a predatory bacterial species.

23. The bacterial composition of claim 22, wherein the predatory bacteria is Bdellovibrio bacteriovorus, and wherein Bdellovibrio bacteriovorus is genetically engineered to target specific pathogenic bacteria.

24. The bacterial composition of claim 22, wherein the bacterial comprises 10⁶ to 10¹² CFU of predatory bacteria.

25. (canceled)
